(19) United States
(12) Patent Application Publication
(10) Pub. No.: US 2025/0208112 A1
(43) Pub. Date: Jun. 26, 2025

(54) COVER-COMPATIBLE WATER ANALYZER

(71) Applicant: ZODIAC POOL CARE EUROPE, Belberaud (FR)

(72) Inventor: Simon Duffaut, Castelginest (FR)

(73) Assignee: ZODIAC POOL CARE EUROPE, Belberaud (FR)

(21) Appl. No.: 18/989,399

(22) Filed: Dec. 20, 2024

Related U.S. Application Data
(60) Provisional application No. 63/614,504, filed on Dec. 22, 2023.

(51) Int. Cl. G01N 33/18; E04H 4/06; H04W 4/38

(52) U.S. Cl. CPC G01N 33/1886; H04W 4/38; E04H 4/06

(57) ABSTRACT

A water analyzer for a swimming pool or spa includes a submergible portion and a projecting portion. The projecting portion extends upwards from the submergible portion and defines a top end of the water analyzer. A maximum thickness of the projecting portion may be less than a maximum thickness of the submergible portion. Additionally, or alternatively, the projecting portion does not overlap a pool cover

### REFERENCE TO RELATED APPLICATION

This application claims the benefit of and priority to U.S. Provisional Patent Application No. 63/614,504, filed on Dec. 22, 2023, and entitled COVER-COMPATIBLE WATER ANALYZER, the content of which is hereby incorporated by reference in its entirety.

### FIELD OF THE INVENTION

The invention relates to systems, apparatuses and methods for fluid analyzers, such as water analyzers for swimming pools and spas, and more particularly, although not necessarily exclusively, to a cover-compatible water analyzer.

### BACKGROUND OF THE INVENTION

Water analyzers may be utilized with water-containing vessels such as swimming pools or spas (hereinafter “swimming pools”) to monitor or detect characteristics of water of the swimming pools such as water temperature, oxidative reduction potential (ORP), pH, salinity, water level, etc. Traditional water analyzers may float within the swimming pool at the waterline but typically cause or have issues with pool covers. For example, water analyzers at the waterline may prevent a pool cover from closing and/or may cause deployment issues with the pool cover by obstructing a deployment of the pool cover. While some water analyzers may go under the pool cover when the pool cover is deployed, such water analyzers suffer from connectivity issues.

### SUMMARY

Embodiments covered by this patent are defined by the claims below, not this summary. This summary is a high-level overview of various embodiments and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to some embodiments, a water analyzer for a swimming pool or spa includes a submergible portion and a projecting portion. The projecting portion extends upwards from the submergible portion and defines a top end of the water analyzer. In certain aspects, a maximum thickness of the projecting portion is less than a maximum thickness of the submergible portion.

According to various embodiments, a water analyzer for a swimming pool or spa includes a submergible portion and a projecting portion, where the projecting portion extends upwards from the submergible portion and defines a top end of the water analyzer. In various cases, a maximum thickness of the projecting portion is less than a gap defined between a pool cover and a wall of the swimming pool or spa.

According to certain embodiments, a water analyzer for a swimming pool or spa includes a submergible portion and a projecting portion, and the projecting portion extends upwards from the submergible portion and defines a top end of the water analyzer. In various aspects, the projecting portion does not overlap a pool cover, at least when the pool cover is in a deployed configuration and the water analyzer is in the swimming pool or spa.

According to various embodiments, a method of analyzing water of a swimming pool or spa includes providing a water analyzer within the swimming pool or spa such that at least a portion of a submergible portion of the water analyzer is within water of the swimming pool or spa and causing deployment of a pool cover over the swimming pool or spa. A projecting portion of the water analyzer extending from the submergible portion may be retained within a gap between the pool cover and a wall of the swimming pool or spa.

Various implementations described herein can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The specification makes reference to the following appended figures, in which use of like reference numerals in different figures is intended to illustrate like or analogous components.

FIG. 1 illustrates a pool system with a water analyzer according to embodiments.

FIG. 2 is another view of the pool system of FIG. 1.

### DESCRIPTION OF THE INVENTION

Described herein are systems and methods involving a cover-compatible water analyzer for a swimming pool or spa. In certain embodiments, the water analyzers described herein may allow for a pool cover to be deployed and/or closed over the swimming pool or spa while the water analyzer is within the swimming pool or spa while maintaining connectivity and/or performance of the water analyzer.

In various embodiments, the water analyzer includes a submergible portion and a projecting portion. The projecting portion may define a top end of the water analyzer and project above the pool cover when the pool cover is deployed. In various embodiments, a maximum thickness of the projecting portion is less than a maximum thickness of the submergible portion. In certain embodiments, a maximum thickness of the projecting portion may be less than a standard gap size recommended between the closed pool cover and a wall of the pool.

Optionally, a communication module or means such as but not limited to an antenna may be within the projecting portion. The systems and methods described herein may allow for a floating water analyzer to provide data (e.g., measured information about one or more characteristics of the swimming pool or spa) and/or receive data from an external device or source even if the pool cover is closed. Wireless communication may be various types of communication as desired, such as but not limited to, WiFi, Bluetooth, Bluetooth Low Energy (BLE), cellular, Zigbee, LPWAN, NFC, and/or as otherwise desired. In certain embodiments, maintaining connectivity performance may save energy and/or reduce energy consumption by the water analyzer, thereby allowing the water analyzer to require charging less often compared to traditional approaches.

Various other benefits and advantages may be realized with the systems and methods provided herein, and the aforementioned advantages should not be considered limiting.

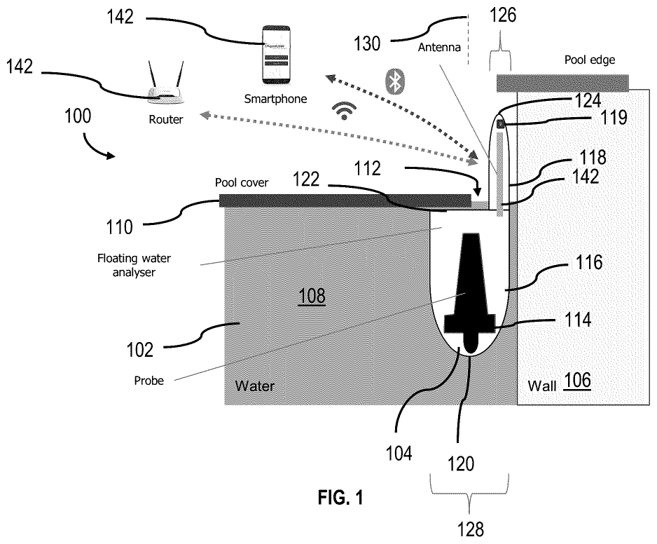

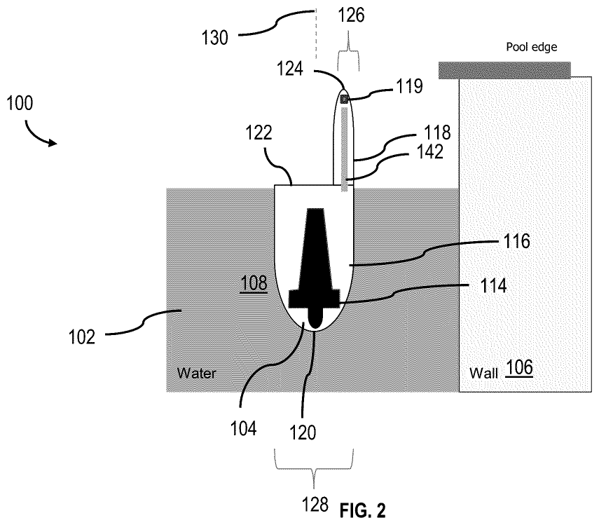

FIGS. 1 and 2 illustrate a pool system 100 according to embodiments. The pool system 100 generally includes a pool 102 and a water analyzer 104.

The pool 102 may have various features, arrangements, shapes, and profiles as desired. As illustrated in FIGS. 1 and 2, in various embodiments, the pool 102 may have one or more walls 106. Optionally, the pool 102 may include a pool cover 110 for selectively covering the pool 102 (e.g., when the pool 102 is not in use). The pool cover 110 may be movable between a stowed (or uncovering) position and a deployed (or covering) position relative to the pool 102 using various techniques or mechanisms as desired, including automatically and/or manually. FIG. 1 illustrates the pool cover 110 in the deployed position, and FIG. 2 omits the pool cover 110 representing the pool cover 110 in the stowed position. As illustrated in FIG. 1, in the deployed position, a gap 112 is defined between an edge of the pool cover 110 and the wall 106. Optionally, in various embodiments, the gap 112 may be a standard gap size recommended by the pool industry between the edge and the wall 106 of about 2.5 cm.

The water analyzer 104 may be provided within water 108 of the pool 102 and includes one or more probes or sensors 114 for detecting or measuring one or more characteristics of the water 108. The one or more water characteristics may include, but are not limited to, oxidation-reduction potential (ORP), pH, water temperature, conductivity, salinity, water level, other types of water sensors, and/or combinations thereof. In some embodiments, the water analyzer 104 may include a plurality of sensors 114 for detecting a plurality of water characteristics. Optionally, the water analyzer 104 may include additional sensors for detecting or measuring information about components of the water analyzer 104 (e.g., battery status, communication status), and/or environmental information (e.g., wind force, air temperature, wave force, etc.).

As illustrated in FIGS. 1 and 2, the water analyzer 104 generally includes a submergible portion 116 and a projecting portion 118. The water analyzer 104 may include various devices or mechanisms such that the water analyzer 104 is buoyant and may float within the pool 102.

The submergible portion 116 includes a bottom end 120, which may be the bottom end of the water analyzer 104, and a top end 122. In certain embodiments, and as illustrated in FIGS. 1 and 2, at least the bottom end 120 is submerged within the water 108 when the water analyzer 104 is provided within the pool 102. Optionally, the top end 122 may be submerged, although it need not be in other embodiments. As a non-limiting example, FIG. 2 illustrates the top end 122 above a waterline of the pool 102. As illustrated in FIGS. 1 and 2, the one or more sensors 114 may be housed by and/or retained on the submergible portion 116 of the water analyzer 104. In other embodiments, the one or more sensors 114 may be provided at various locations on and/or within the water analyzer 104 as desired.

The projecting portion 118 of the water analyzer 104 extends upwards from the top end 122 of the submergible portion 116. In certain embodiments, the projecting portion 118 has a height such that the projecting portion 118 extends above a waterline of the pool 102 when the water analyzer 104 is provided within the pool 102. The projecting portion 118 includes a top end 124, which may be the top end of the water analyzer 104.

In certain embodiments, and as illustrated in FIGS. 1 and 2, the projecting portion 118 is offset relative to a center axis 130 of the submergible portion 116. The offset projecting portion 118 may facilitate providing information to a user as described in detail below. In some embodiments, the offset projecting portion 118 may facilitate the projecting portion 118 extending above a pool cover when the pool cover is deployed, thereby enabling communication with the water analyzer 104 regardless of whether the pool cover is stowed or deployed. Optionally, the offset projecting portion 110 may passively promote movement of the water analyzer 104 within the pool 102 (e.g., by creating an unbalanced weight distribution above the water, optionally allowing for wind, waves, water flow, etc. to push or otherwise cause movement of the water analyzer 104).

As illustrated in FIGS. 1 and 2, in certain embodiments, a maximum thickness 126 of the projecting portion 118 is less than a maximum thickness 128 of the submergible portion 116. In some embodiments, the relative maximum thickness 126 of the projecting portion 118 and the positioning of the projecting portion 118 (e.g., offset or as otherwise desired) may facilitate providing information to a user, such as but not limited to when the pool cover is deployed. In some embodiments, the maximum thickness 126 of the projecting portion 118 is less than the thickness of the gap 112.

In certain embodiments, the water analyzer 104 includes a communication module and/or means, such as but not limited to one or more antennas 142. The antennas 142 and/or other communication module and/or means may be provided at various locations as desired. In certain embodiments, the antennas advantageously may be provided within the projecting portion 118, thereby facilitating communication with the water analyzer 104 even when the pool cover is deployed. The communication module and/or means may enable the water analyzer 104 to receive and/or send data to one or more external devices 144 via wireless communication. The wireless communication supported by the communication module and/or means may include, but is not limited to, WiFi, Bluetooth, Bluetooth Low Energy (BLE), cellular, Zigbee, LPWAN, NFC, and/or others. In some embodiments, when the one or more antennas 142 are included, the one or more antennas 142 may extend upwards and/or in a vertical direction relative to the submergible portion 116.

Optionally, the water analyzer 104 may include one or more indicators 119 for providing information to a user. The information provided to the user may be information about one or more water characteristics (e.g., as obtained by the one or more sensors of the water analyzer), information about components of the water analyzer (e.g., battery status, communication status), and/or environmental information (e.g., wind force, air temperature, wave force, etc.). The indicator 119 may be various devices, mechanisms, or features for providing information to a user locally on the water analyzer. As non-limiting examples, the indicator 119 may be a screen, display, one or more light sources, a color roll, electronic ink, other devices and/or features that may or may not require energy consumption to provide information to the user, combinations thereof, and/or other devices. The indicator 119 may be provided on the projecting portion 118 and/or the submergible portion 116 of the water analyzer as desired. In one non-limiting example, the water analyzer 104 includes a light source and/or a screen on the projecting portion 118 and/or at a top of the submergible portion 116 as the indicator 119.

The projecting portion 118 having the maximum thickness 126 less than the thickness of the gap 112 and/or the maximum thickness 128 of the submergible portion 116, and the projecting portion 118 being offset from the center axis 130 may allow for the pool cover 110 to be in the deployed position (see FIG. 1) while the projecting portion 118 extends above the waterline. As illustrated, when the pool cover 110 is in the deployed position, the projecting portion 118 may extend upwards relative to the pool cover 110. In certain aspects, the pool cover 110 may at least partially overlap or cover the submergible portion 116.

In certain embodiments, the projecting portion 118 does not overlap the pool cover 110 in the deployed position, which may provide improved positioning of the water analyzer 104, and the water analyzer 104 need not account for accommodating waves in the pool 102, defects in the pool cover 110, etc. Moreover, the projecting portion 118 not overlapping the pool cover 110 may provide improved mobility of the water analyzer 104 and minimize engagement between the pool cover 110 and the water analyzer 104.

In certain embodiments, the water analyzer 104 with the projecting portion 118 need not depend on the type of cover, and the water analyzer 104 may be compatible with a variety of cover types (e.g., regardless of thickness of the cover, material of the cover, hardness of the cover, etc.). The water analyzer 104 described herein may have improved connectivity range (e.g., due to the height of the projecting portion 118 and/or the antenna thereon), and the water analyzer 104 may be easier to remove from water of the pool when the cover 110 is deployed compared to traditional approaches.

As illustrated in FIG. 1, the projecting portion 118 within the gap 112 and/or extending above the pool cover 110 when the pool cover 110 is deployed may allow for the water analyzer 104 to maintain connectivity with the external devices 144 and/or maintain performance. As non-limiting examples, even when the pool cover 110 is deployed, the water analyzer 104 may continue to send water characteristic information obtained by the sensors 114 to the one or more external devices 144 and/or receive information from the external devices 144. Maintaining connectivity and performance may allow for the water analyzer to save energy/improve energy consumption, thereby allowing the water analyzer 104 to be charged less frequently compared to traditional approaches.

Various other benefits and advantages may be realized with the systems and methods provided herein, and the aforementioned advantages should not be considered limiting.

Exemplary concepts or combinations of features of the invention may include:
A. A water analyzer for a swimming pool or spa, the water analyzer comprising a submergible portion and a projecting portion, wherein the projecting portion extends upwards from the submergible portion and defines a top end of the water analyzer, and wherein a maximum thickness of the projecting portion is less than a maximum thickness of the submergible portion.
B. A water analyzer for a swimming pool or spa, the water analyzer comprising a submergible portion and a projecting portion, wherein the projecting portion extends upwards from the submergible portion and defines a top end of the water analyzer, and wherein a maximum thickness of the projecting portion is less than a gap defined between a pool cover and a wall of the swimming pool or spa.
C. A water analyzer for a swimming pool or spa, the water analyzer comprising a submergible portion and a projecting portion, wherein the projecting portion extends upwards from the submergible portion and defines a top end of the water analyzer, and wherein the projecting portion does not overlap a pool cover.
D. The water analyzer of any preceding or subsequent statement or combination of statements, wherein the projecting portion comprises a communication module.
E. The water analyzer of any preceding or subsequent statement or combination of statements, wherein the communication module is configured to communicate via a wireless protocol such as but not limited to Wifi, Bluetooth, cellular, Zigbee, NFC, and/or a low-power wide area network (LPWAN).
F. The water analyzer of any preceding or subsequent statement or combination of statements, wherein the projecting portion comprises an antenna, and wherein the water analyzer is configured to wirelessly communicate with an external device via the antenna.
G. The water analyzer of any preceding or subsequent statement or combination of statements, wherein the antenna extends vertically relative to the submergible portion.
H. The water analyzer of any preceding or subsequent statement or combination of statements, wherein the submergible portion comprises a probe or sensor configured to measure or detect at least one characteristic of water of the swimming pool or spa.
I. The water analyzer of any preceding or subsequent statement or combination of statements, wherein the at least one characteristic of water comprises at least one of temperature, ORP, pH, salinity, water level, or other characteristics as desired.
J. The water analyzer of any preceding or subsequent statement or combination of statements, wherein the submergible portion comprises a top end and a bottom end, wherein the projecting portion extends upwards from the top end of the submergible portion.
K. The water analyzer of any preceding or subsequent statement or combination of statements, wherein at least the bottom end of the submergible portion is submerged when the water analyzer is within the swimming pool or spa.
L. The water analyzer of any preceding or subsequent statement or combination of statements, wherein the projecting portion is offset from a central axis of the submergible portion.
M. The water analyzer of any preceding or subsequent statement or combination of statements, wherein the water analyzer is buoyant and configured to float within the swimming pool or spa.
N. A method of analyzing water of a swimming pool or spa, the method comprising providing a water analyzer according to any preceding or subsequent statement or combination of statements within the swimming pool or spa.
O. A method of analyzing water of a swimming pool or spa, the method comprising:
   i. providing a water analyzer within the swimming pool or spa such that at least a portion of a submergible portion of the water analyzer is within water of the swimming pool or spa; and
   ii. causing deployment of a pool cover over the swimming pool or spa, wherein a projecting portion of the water analyzer extending from the submergible portion is retained within a gap between the pool cover and a wall of the swimming pool or spa.
P. The method of any preceding or subsequent statement or combination of statements, further comprising sending data to and/or receiving data from the water analyzer while the pool cover is deployed.
Q. The method of any preceding or subsequent statement or combination of statements, wherein the pool cover at least partially overlaps the submergible portion of the water analyzer.

These examples are not intended to be mutually exclusive, exhaustive, or restrictive in any way, and the invention is not limited to these example embodiments but rather encompasses all possible modifications and variations within the scope of any claims ultimately drafted and issued in connection with the invention (and their equivalents). For avoidance of doubt, any combination of features not physically impossible or expressly identified as non-combinable herein may be within the scope of the invention. Further, although applicant has described devices and techniques for use principally with swimming pools and spas, persons skilled in the relevant field will recognize that the present invention conceivably could be employed in connection with other objects and in other manners. Finally, references to “pools” and “swimming pools” herein may also refer to spas or other water containing vessels used for recreation, training, or therapy.

That which is claimed:

1. A water analyzer for a swimming pool or spa, the water analyzer comprising a submergible portion and a projecting portion, wherein the projecting portion extends upwards from the submergible portion and defines a top end of the water analyzer, and wherein a maximum thickness of the projecting portion is less than a maximum thickness of the submergible portion.

2. The water analyzer of claim 1, wherein the water analyzer is buoyant and configured to float within the swimming pool or spa.

3. The water analyzer of claim 1, wherein the projecting portion comprises a communication module.

4. The water analyzer of claim 3, wherein the communication module is configured to communicate via a wireless protocol comprising at least one of WiFi, Bluetooth, cellular, Zigbee, NFC, and/or a low-power wide area network (LPWAN).

5. The water analyzer of claim 1, wherein the projecting portion comprises an antenna, and wherein the water analyzer is configured to wirelessly communicate with an external device via the antenna.

6. The water analyzer of claim 5, wherein the antenna extends vertically relative to the submergible portion.

7. The water analyzer of claim 1, wherein the submergible portion comprises a probe or sensor configured to measure or detect at least one characteristic of water of the swimming pool or spa.

8. The water analyzer of claim 7, wherein the at least one characteristic of water comprises at least one of temperature, ORP, pH, salinity, or water level.

9. The water analyzer of claim 1, wherein the submergible portion comprises a top end and a bottom end, wherein the projecting portion extends upwards from the top end of the submergible portion.

10. The water analyzer of claim 9, wherein at least the bottom end of the submergible portion is submerged when the water analyzer is within the swimming pool or spa.

11. The water analyzer of claim 9, wherein the projecting portion is offset from a central axis of the submergible portion.

12. The water analyzer of claim 1, wherein the maximum thickness of the projecting portion is less than a gap defined between a pool cover and a wall of the swimming pool or spa.

13. A water analyzer for a swimming pool or spa, the water analyzer comprising a submergible portion and a projecting portion, wherein the projecting portion extends upwards from the submergible portion and defines a top end of the water analyzer, and wherein the projecting portion does not overlap a pool cover.

14. The water analyzer of claim 13, wherein the projecting portion comprises an antenna, and wherein the water analyzer is configured to wirelessly communicate with an external device via the antenna.

15. The water analyzer of claim 13, wherein the submergible portion comprises a probe or sensor configured to measure or detect at least one characteristic of water of the swimming pool or spa.

16. The water analyzer of claim 13, wherein the submergible portion comprises a top end and a bottom end, wherein the projecting portion extends upwards from the top end of the submergible portion, wherein the projecting portion is offset from a central axis of the submergible portion.

17. The water analyzer of claim 16, wherein a maximum thickness of the projecting portion is less than a gap defined between a pool cover and a wall of the swimming pool or spa and less than a maximum thickness of the submergible portion.

18. A method of analyzing water of a swimming pool or spa, the method comprising:
providing a water analyzer within the swimming pool or spa such that at least a portion of a submergible portion of the water analyzer is within water of the swimming pool or spa; and
causing deployment of a pool cover over the swimming pool or spa, wherein a projecting portion of the water analyzer extending from the submergible portion is retained within a gap between the pool cover and a wall of the swimming pool or spa.

19. The method of claim 18, further comprising sending data to and/or receiving data from the water analyzer while the pool cover is deployed.

20. The method of claim 18, wherein the pool cover at least partially overlaps the submergible portion of the water analyzer.
